(19) United States
(12) Patent Application Publication
(10) Pub. No.: US 2011/0125143 A1
(43) Pub. Date: May 26, 2011

(54) THERMAL ABLATION DEVICE, CATHETER AND METHOD TO IMPLEMENT A THERMAL ABLATION

(76) Inventors: Patrick Gross, Langensendelbach (DE); Clifford Weiss, Baltimore, MD (US); Anke Weissenborn, Weil am Rhein (DE)

(21) Appl. No.: 12/947,357

(22) Filed: Nov. 16, 2010

(30) Foreign Application Priority Data
Nov. 16, 2009 (DE) 10 2009 053 470.9

(51) Int. Cl. A61B 18/02; A61B 18/04

(52) U.S. Cl. 606/27; 606/1

(57) ABSTRACT

A thermal ablation device for introduction of heat or cold into the body of a patient has a first heating or cooling device to heat or cool the tissue to be ablated, as well as at least one additional cooling or heating arrangement that serves to cool or heat tissue surrounding the tissue to be ablated, and that can be introduced into a natural or latent cavity of the patient.

### BACKGROUND OF THE INVENTION

1. Field of the Invention

The invention concerns a thermal ablation device to introduce heat or cold into the body of a patient, with a first heating or cooling device to heat or cool tissue to be ablated.

2. Description of the Prior Art

Thermal ablation is the obliteration of tissue that is produced by introduction of heat or cold to the tissue. For example, in cryo-ablation, a cryo-ablation catheter is inserted into the body of the patient, the tip of the cryo-ablation catheter is cooled to temperatures below 0° C. and the tissue surrounding the tip of the catheter is thereby irreversibly damaged and thus obliterated. To obliterate tissue by means of the introduction of heat, an ultrasound device (for example) can be used that is HIFU-capable (HIFU=high intensity focused ultrasound). In HIFU, the energy injection is concentrated by a particular formation of the sound beam; the tissue of a patient is thereby heated in a delimited region and is thereby obliterated.

Thermal ablation devices thus are based on two different principles. As in HIFU, the energy injection can be defined via the shape of the energy field. Microwave-based ablation devices also fall into this embodiment.

In a further embodiment, the introduction of heat or cold proceeds directly from a corresponding device and is limited to a specified region around the device. In addition to the cryo-ablation catheter, laser and radio-frequency probes are also examples of these types of devices.

One problem in these ablation devices is that the introduction of heat or cold cannot be limited or adjusted precisely to the tissue to be ablated. Tissue that surrounds the tissue to be ablated thus may also be damaged by the ablation device. This is particularly problematic when the surrounding tissue or portions of this have important functional tasks. For example, one problem in the treatment of prostate tumors is that the nerves running on both sides of the prostate capsule can be damaged given use of a thermal ablation device, and this can lead to erectile dysfunction after the procedure.

### SUMMARY OF THE INVENTION

An object of the present invention is to provide a thermal ablation device in which the region affected by the introduction of heat or cold can be better delimited, so that tissue that is not to be ablated can be better protected from destruction or damage.

This object is achieved by thermal ablation device according to the invention that it has at least one additional cooling or heating arrangement that serves to cool or heat the tissue surrounding the tissue to be ablated, and that can be introduced into a natural or latent cavity of the patient.

According to the invention, the thermal effect of the ablation device is canceled or is at least counteracted by a second device in one or more tissue regions that are not to be ablated. If a cryo-ablation catheter is used as a thermal ablation device, a heating arrangement is used to protect the surrounding tissue. Given the use of an ablation device operating by heat introduction—for example HIFU—the tissue that surrounds the tissue to be ablated is correspondingly cooled by a cooling arrangement.

In order to be able to realize the use of the additional cooling or heating arrangement so as to be as non-invasive as possible, cooling or heating arrangement are used that can be introduced into a natural or latent cavity of the patient. Natural cavities are cavities that already exist in vivo, for example the space within the bladder, the inside of the intestine, the inside of the stomach or even the blood vessels. A latent cavity is a cavity that does not exist in the natural state of the patient but that can be produced without cutting or otherwise injuring the tissue. The pleural cavity between the parietal pleura and visceral pleura is such a latent cavity. Regions between organs can also be used as a latent cavity, wherein the cooling or heating arrangement is then to be introduced into the body of the patient in a minimally invasive manner, for example by means of an endoscopic procedure.

The additional cooling or heating arrangement can advantageously have a larger heat capacity than the tissue surrounding the tissue to be ablated. The cooling or heating arrangement serves to directly protect the tissue that surrounds the tissue to be ablated. The cooling or heating arrangement must accordingly ensure a corresponding energy introduction or energy extraction. If this capability increases with an increasing heat capacity, the cooling or heating arrangement can accordingly cool or heat the surrounding tissue for a longer period of time than given the use of an arrangement without such a large heat capacity.

The additional cooling or heating arrangement can advantageously exhibit a lower or higher temperature than the body of the patient upon introduction into the body. The temperature difference can be less than 5° C. In the event of a higher temperature, the heating arrangement then acts in the manner of a hot-water bottle. The thermal energy (or also the amount of heat) stored in the heating arrangement is used to warm the surrounding tissue until the temperatures of the heating arrangement and the surrounding tissue have equalized.

The temperature of the surrounding tissue and that of the heating arrangement continues to drop as a result of the introduction of cold by the first cooling device present in this case, even below the normal temperature of the surrounding tissue. However, treatments with thermal ablation devices are time-limited. The thermal energy of the heating arrangement thus must only be sufficient to prevent a drop of the temperature of the surrounding tissue below a critical value for the time duration of the introduction of cold. Naturally, the temperature of the surrounding tissue thus does not need to be kept at a normal temperature; only a drop below a temperature at which the surrounding tissue is irreversibly damaged or destroyed is to be avoided. In the event that the heating arrangement contacts the tissue to be ablated, the effect of the thermal ablation device is reduced in this region. The introduction of heat by the ablation device and the introduction of cold by the additional cooling arrangement are accordingly to be matched so that the surrounding tissue is protected without hindering the obliteration of the tissue to be ablated.

In the event that a HIFU device or any other device based on an introduction of heat is used as an ablation device, the additional arrangement is naturally a cooling arrangement that cools the surrounding tissue.

The additional cooling or heating arrangement can particularly advantageously be fashioned to supply a medium, in particular a liquid or a gas. Liquids and gases allow a fast heat exchange between the liquid or the gas and the surrounding tissue, so the heat exchange is optimized. In addition to the introduction of pure liquids and pure gases, the introduction of liquid/gas mixtures and even liquid/solid mixtures is also conceivable. The solid body portions (for example in the form of small spheres) can be soluble in the fluid.

In one embodiment the additional cooling or heating arrangement can be a balloon that can be filled with a medium, in particular a liquid or a gas. The use of a balloon prevents the direct contact of the medium with the body of the patient. Media that are inherently toxic to patients can accordingly also be used for cooling or heating. The medium used for cooling can also be completely removed from the body again without any residues remaining in the body (which residues are then to be metabolized by the body). In particular, a balloon has a ductile wall so it can be flexibly adapted to different patients. For example, if a balloon is inserted into the rectum of a patient, depending on the patient the rectum exhibits different diameters. Given the use of a balloon, this can expand differently due to the fill quantity and thus can be adapted to the measurements of the patient.

The wall thickness of the balloon can advantageously vary. It is thereby achieved that the heat exchange between the balloon and the tissue surrounding the balloon proceeds differently. For example the balloon can possess a thin wall thickness on one side and a thicker wall thickness on the other side. The wall with the thinner wall thickness is thereby facing towards the tissue that surrounds the tissue to be ablated. In contrast to this, the side with the thicker wall is positioned towards the tissue to be ablated in order to not negatively affect the first heating or cooling device. In principle, all possible shapes with alternating wall thicknesses are thereby conceivable.

The balloon can particularly advantageously exhibit a circular thinning of the wall thickness. The introduction of heat or cold to the surrounding tissue can thereby ensue point-by-point. The wall thickness of the balloon can also exhibit circular thinning at multiple locations or can also exhibit only one circular thickening, wherein the circular thickening then faces towards the tissue to be ablated.

In one embodiment the additional cooling or heating arrangement can possess at least one hollow needle. Cavities in the body of the patient that are not accessible via a bodily orifice can also be reached with hollow needles. Since the medium used for cooling or heating comes into direct contact with the tissue, in no case may it have a severely toxic effect and should be resorbable by the body. In particular, sodium chloride solution is a suitable medium in this case.

In a further embodiment, the additional cooling or heating arrangement can be fashioned as a catheter. In the simplest embodiment the catheter has a carbon cavity into which a medium can be introduced via an access, so the catheter itself is cooled or warmed.

In one embodiment, the catheter can have, in the distal region, a balloon with which a natural cavity in the body of the patient can be sealed. This balloon is located below the tip of the catheter, and the tip of the catheter has access to the cavity in the body of the patient. The catheter can advantageously have at the distal end one or more openings for the introduction of a medium—in particular a liquid or gas—into the cavity in the body of the patient. The natural cavities in the body of the patient are not all completely closed. For example, the uterus has an opening to the vagina that can be sealed by the balloon in order to ensure the retention of the medium in the uterus. The openings at the catheter tip then enable the introduction of the medium into the uterus, which is sealed there due to the balloon. After conducting the ablation, the medium can easily be removed again by reducing the balloon volume and the subsequent removal of the catheter.

The additional cooling or heating arrangement can have at least one inflow and at least one outflow via which the medium—in particular the liquid or the gas—can be exchanged in the cavity or in the balloon. The capability of the cooling or heating arrangement to introduce cold or heat is increased by exchanging the medium. Not only is heat energy (in this case both the capability for heat introduction and the capability for heat extraction are meant) provided that is possessed by the medium upon introduction into the body, but also the cooling or heating arrangement can be used to cool or heat for a longer period of time The heat capacity can be virtually increased in this manner. The actual heat capacity of the medium that is present naturally does not change, it only appears greater due to the exchange. The heat exchange that is realized by means of convection, as in the example of the balloon and the catheter, is improved by the forced convection.

The designs of the inflow and outflow are respectively dependent on the embodiment of the cooling or heating arrangement. Given the use of a catheter, the inflow and the outflow can respectively be fashioned as a lumen in the catheter. These can be thermally insulated. The thermal insulation prevents heat exchange between the medium and the body of the patient on the route to the catheter. Heat exchange with the catheter itself also can be avoided. A maximum heat introduction or heat extraction thus can be realized in the cavity.

In contrast to this, given an embodiment of the cooling or heating arrangement as a hollow needle, two hollow needles are to be used, wherein one operates as an inflow and the other operates as an outflow. For example, the hollow needles can be inserted into the bladder on opposite sides, so the introduced and exchanged medium protects the bladder wall. A balloon can also have an inflow and an outflow, designed as openings in the balloon wall that are connected with extracorporeal devices via tubes.

In a further embodiment, the additional cooling or heating arrangement can have a nozzle with which the medium—in particular the fluid or the gas—can be sprayed onto the wall of the balloon or the cavity. The heating or cooling effect thus can be achieved in different ways. If the medium is atomized (nebulized) such that an adiabatic expansion occurs, the medium is cooled during the expansion process. The nozzle for atomization can be attached at the end of a hollow needle, at the opening of a catheter and at an inflow of a balloon. Given use of a hollow needle or a catheter, the surrounding tissue is directly cooled via the atomized medium; given use of a balloon, the surrounding tissue is cooled indirectly via the cooling of the balloon wall.

A chemical reagent that produces a release of heat or an extraction of heat due to a chemical reaction can advantageously be used as a medium. The chemical reagent enables a removal or introduction of heat energy over a longer period of time that can be implemented without exchanging the medium. In this way a large quantity of heat energy can be stored, both in the sense of a heat release and a heat extraction, without the heating arrangement itself having to be significantly heated or cooled for this purpose.

In a further embodiment, the additional cooling or heating arrangement can be a metal plate. The shape of the metal plate can be cuboid or any arbitrary shape. Metal has a high specific heat capacity, which is advantageous overall for the heat capacity of the metal plate. In particular, the metal plate can be adapted to specific organs or regions of organs, so it is possible to heat or cool specific regions in the body of the patient only in a predetermined area.

The additional cooling arrangement can particularly advantageously be or include a Peltier cooler with which the wall of the cavity or the balloon can be directly or indirectly cooled by an introduced medium, in particular a liquid or a gas. The heat removal from the surrounding tissue is based on the fact that a heat exchange occurs between the cooling arrangement and the surrounding tissue. Upon bringing together two bodies (thus the surrounding tissue and the cooling arrangement), a mixture temperature normally arises in the bodies due to the heat exchange processes. This mixture temperature depends on the mass of the body, the respective, specific heat capacities and the initial temperatures. This mixture temperature naturally lies between the initial temperatures of the two bodies. The heat energy that can be extracted from the surrounding tissue is thus limited by the mass and the specific heat capacity of the coolant as well as its initial temperature. The initial temperature itself should not be too low, since otherwise the surrounding tissue is damaged by the additional cooling arrangement. The heat energy that can be extracted in total from the surrounding tissue is thus also limited. This limitation can be improved by a continuous removal of heat energy. The Peltier cooler can be placed either directly at the wall of the cavity or balloon or can merely project into the medium in order to cool the medium.

Alternatively, the additional heating arrangement can be or include a heatable resistor with which the wall of the cavity or of the balloon can be heated directly or indirectly via an introduced medium, in particular a liquid or a gas. The resistor represents the counterpart to the Peltier cooler; its use occurs in an analogous manner. In particular, the additional cooling or heating arrangement can include both a Peltier cooler and a heatable resistor. In the case of a balloon, this is filled with a medium and moreover has another yet another resistor as well as a Peltier cooler inside the balloon which are respectively in contact with the medium. Depending on the usage purpose, either the Peltier cooler or the resistor is then operated to cool or heat the medium. It is thereby possible to provide a single item that can be used both as an additional cooling arrangement and as an additional heating arrangement. The number of instruments to be used can thereby be reduced. This is particularly advantageous if different and respectively adapted cooling or heating arrangements are used for different cavities and the number of cooling and heating arrangements can thus be halved.

The cooling medium can advantageously be a liquid medium on the wall of the cavity or of the balloon. The liquid can be brought to the wall of the cavity via both a hollow needle and a catheter. The surrounding tissue is heated by the ablation device, whereby the liquid film is evaporated. Upon this evaporation heat is drawn from the surrounding tissue or the wall of the balloon, so this is cooled. After the vaporization of the liquid a new liquid film is to be established at the wall of the cavity or of the balloon in order to maintain the evaporation process. In this case the provision of an inflow and an outflow is advantageous, wherein the liquid is introduced via the inflow and the vaporized gas is removed via the outflow.

In addition, the invention concerns a catheter for a thermal ablation device comprising a first heating or cooling device to warm or cool tissue of a patient that is to be ablated. The catheter is characterized in that it possesses an additional cooling or heating arrangement that acts in a non-ablative manner in operation, which additional cooling or heating arrangement serves to cool or heat tissue that surrounds the tissue to be ablated.

The cooling or heating arrangement can be located in the region of the distal end of the catheter. The cooling or heating arrangement can accordingly be located at the tip of the catheter but can also be at a distance from the tip of the catheter. The cooling or heating arrangement can thus act at a point (assuming the tip of the catheter) and can also act to one side or over an area.

The catheter can advantageously possess a balloon that is variable in volume and at least one first lumen with which the balloon can be filled with a medium, in particular a liquid or a gas. The balloon can be located at the tip of the catheter or also to one side. It can be (but does not have to be) suitable for sealing the cavity in the body of the patient. The balloon is filled by means of the first lumen; the surrounding tissue is thus protected via the balloon. In an extension of the aforementioned embodiments, this is arranged at the catheter and can be filled via a lumen of the catheter.

The catheter can have at least one second lumen, wherein the at least one first lumen can be used as an inflow and the at least one second lumen can be used as an outflow. An inflow and an outflow for the balloon are realized with the first and second lumen, wherein additional lumens are also conceivable. In addition to the cooling or the heating of the surrounding tissue by means of the balloon arranged at the catheter, at least one opening and/or one nozzle with which the surrounding tissue is wetted with a liquid film can be provided at the catheter. A cooling effect as described above occurs upon vaporization of the liquid film. As a second function, the balloon that cools the surrounding tissue can also seal a natural cavity in the patient, which cavity can be filled by means of at least two additional lumens that respectively act as inflow and outflow. Two lumens are thus respectively provided for the media exchange in the balloon and in the cavity.

The invention also concerns a method to implement a thermal ablation of a tissue region of a patient, wherein the tissue to be ablated is heated or cooled with a first heating or cooling device. The method is characterized by, in addition to the first heating or cooling device of the ablation device, at least one further cooling or heating arrangement is used that cools or heats the tissue surrounding the tissue to be ablated and that is inserted into a natural or latent cavity of the patient. The method can also be developed within the scope of the aforementioned embodiments in order to achieve the respective advantages.

### BRIEF DESCRIPTION OF THE DRAWINGS

FIG. 1 is a basic representation of an ablation device.

FIG. 2 is a basic representation of an additional cooling or heating arrangement in a first embodiment.

FIG. 3 is a basic representation of an additional cooling or heating arrangement in a second embodiment.

FIG. 4 shows a balloon in a first embodiment.

FIG. 5 shows a balloon in a second embodiment.

FIG. 6 shows a catheter in a first embodiment.

FIG. 7 shows a catheter in a second embodiment.

FIG. 8 shows a catheter in a third embodiment.

FIG. 9 shows a thermally insulated lumen.

FIG. 10 shows a catheter in a third embodiment.

FIG. 11 shows a balloon in a third embodiment.

FIG. 12 shows a metal plate in a first embodiment.

FIG. 13 shows a metal plate in a second embodiment.

FIG. 14 shows a metal plate in a third embodiment.

FIG. 15 is a basic representation of a catheter with a balloon in a first embodiment.

FIG. 16 is a basic representation of a catheter with a balloon in a second embodiment.

FIG. 17 shows the arrangement of a hollow needle at a latent cavity.

FIG. 18 shows a catheter in a fifth embodiment.

FIG. 19 shows a balloon in a fourth embodiment.

### DESCRIPTION OF THE PREFERRED EMBODIMENTS

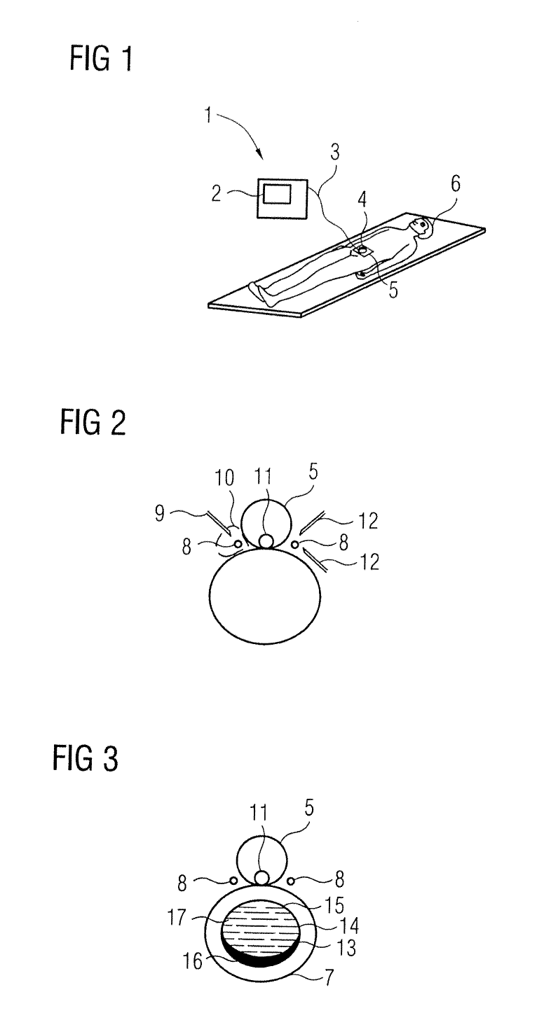

FIG. 1 shows an ablation device 1 comprising a control device 2 and an ultrasound device 3 with an ultrasound head 4. The ultrasound head 4 is fashioned to heat tissue inside the body, for example inside the prostate 5 of the patient 6. The ultrasound device 3 is accordingly an HIFU.

However, tissue regions that can likewise be affected by the introduction of heat by means of the ultrasound head 4 are located directly adjacent to the prostate 5. For example, these are the rectum 7 or the nerves 8. Damage to the nerves 8 can lead to erectile dysfunction of the patient after the procedure. Damage to the rectum 7 is also linked with significant risks to the patient 6. The tissue 11 to be ablated can thereby be a tumor, blastoma (swelling) or any other tissue that is to be obliterated. The use of an HIFU or a thermal ablation device 1 is thereby indicated in all cases in which a local procedure is precluded. The damage to adjacent tissue is thereby accepted since the negative effects on the patient 6 that are caused by this are in every case less severe than the consequences of, for example, a spreading prostate cancer. The hollow needle 9 is provided to protect the tissue surrounding the tissue 11 to be ablated. Cooled sodium chloride solution is introduced into the tissue adjoining the prostate via the hollow needle 9 in order to protect the region 10, and in particular to protect the nerve 8 situated therein. Not only the tissue 11 to be ablated but also the surrounding tissue is heated via the introduction of heat by means of the ultrasound head 4. Damage to the nerves 8 as well as the region 10 is avoided or at least reduced in its effect via the introduction of the sodium chloride solution. In particular, the ablation can be conducted for a longer period of time before damage to the surrounding tissue occurs. In order to design the heat exchange to be as efficient as possible, the sodium chloride solution can be introduced intermittently into the tissue. The warming sodium chloride solution spreads out in the tissue while the cooled sodium chloride solution supplied from the outside is always output in the area of a nerve 8. The cooling of the nerve 8 can thereby be optimized.

Instead of sodium chloride solution, any other liquid or any other gas can also be used that does not have a toxic effect and can be resorbed by the body.

The cooling effect can be improved by, instead of one hollow needle 9, two hollow needles 12 are inserted into the body of the patient 6. The one hollow needle 12 is then used as an inflow via which cooled sodium chloride solution is introduced into the body of the patient while excess and warmed sodium chloride solution is discharged again via the second hollow needle 12. In this embodiment the nerve 8 is continuously flushed by cooler sodium chloride solution, which is why damage to the nerve 8 and the directly adjoining tissue is prevented.

In addition to the nerves 8, the rectum 7 is also a sensitive region in the surroundings of the prostate that is to be protected given ablations at or in the prostate. A corresponding cooling arrangement is shown in FIG. 3. It is thereby a balloon 13 with a wall 14 whose wall thickness varies. The wall 14 is fashioned to be thinner on the segment 15 facing towards the ablating tissue 11 than on the segment 16 facing away from the region 11 to be ablated. The balloon 13 is filled with liquid 17. Due to the different wall thicknesses, the heat exchange between the cooled liquid 17 preferably occurs with the tissue that surrounds the region 11 to be ablated. A heat exchange (wherein what is to be understood by this is both an introduction of heat and a removal of heat) occurs to a reduced extent or even not at all with the tissue that adjoins the segment 16, which is brought about by the insulating effect of the wall 14.

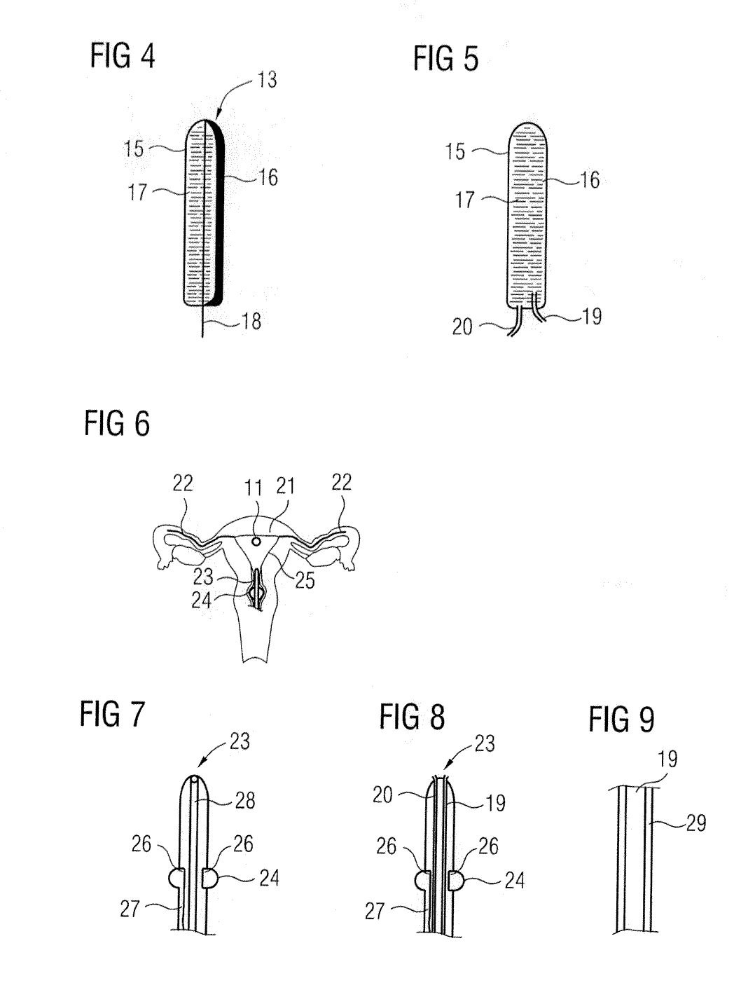

The balloon 13 that is shown in cross section in FIG. 3 can be designed differently in the longitudinal direction. FIG. 4 shows an embodiment in which the variation of the thickness of the wall 14 is executed over the entire length. The guide wire 18 leads into the balloon in order to be able to insert the balloon 13 into the rectum 7 of the patient. Due to the filling with the liquid 17, the balloon 13 itself exhibits an insufficient inherent stability in order to allow the insertion. All liquids can be used as a liquid 17, even liquids that are toxic to and cannot be resorbed by the body. Since the liquid 17 is located in the balloon 13, no contact with the body of the patient arises, which is why the liquid 17 must meet lower requirements than given the use of hollow needles 9, 12. In particular, a chemical reagent that extracts heat from the environment due to a chemical reaction can be used as a liquid 17. For this the liquid can also contain solid bodies—for example in ball form—as chemical reaction partners. Given use of a chemical reagent, the cooling effect of the balloon 13 can be maintained longer than given a simple cooling of the liquid 17 before application of the balloon 13 in the body of the patient 6.

Naturally, multiple cooling or heating arrangement can also be combined with one another in order to achieve an improved heat exchange. For example, the nerves 8 can simultaneously be cooled via hollow needles 9 and 12 and the rectum 7 can be cooled with a balloon 13.

Due to the limitation of the volume of the balloon 13, the quantity of heat that can be removed by means of the balloon 13 is relatively low without using a chemical reagent. This heat quantity that can be absorbed by the balloon 13 is on the one hand to be set in relation to the heat energy that is introduced in total into the tissue by the HIFU, and in particular in relation to the heat energy that is introduced into the surrounding tissue that should be protected with the balloon 13. In order to further increase the heat quantity that can be removed by the balloon 13, the balloon 13 possesses an inflow 19 and an outflow 20. Cooled liquid 17 can be introduced into the balloon 13 via the inflow 19 while already warmed liquid 17 can be conducted out of the balloon via the outflow 20. Heat energy absorbed by the balloon 13 or, respectively, by the liquid 17 is thereby continuous transported away, whereby the balloon 13 can cool the surrounding tissue for an arbitrary length of time. In order to be able to extract the heat from the surrounding tissue at specific points, the segment 15 in which the thickness of the wall 14 is reduced is fashioned in the shape of a circle. The cooling of the surrounding tissue is hereby concentrated at a region that is particularly worthy of protection, whereby the cooling is used in a particularly effective manner.

FIG. 6 shows a further embodiment of the cooling or heating arrangement in the form of a catheter 23. This is inserted into the uterus via the vagina and the cervical channel. A balloon 24 that, in the filled state, seals the cervical channel is located in the region of the distal end of the catheter. A closed cavity that can be filled with a medium is thereby made from the uterus 21. No noteworthy outflow occurs to the fallopian tubes 22.

An embodiment possibility of the catheter 23 is presented in FIG. 7. FIG. 7 shows the catheter 23 in a longitudinal section. The balloon 24 is attached to the catheter 23 over its entire extent and, in the unfilled state, is located in the recess 26i The lumen 27 via which the balloon 24 can be filled with a medium 24 that is stored outside the body is provided to fill said balloon 24. The lumen 28 leads through the catheter 23, via which lumen 28 the medium for cooling or heating the uterus wall is introduced into the uterus 21 after filling the balloon 24. If the uterus 21 is simply filled with an extracorporeally cooled or heated liquid, the heat exchange occurs by means of conduction. The liquid molecules move due to diffusive processes, whereby a natural convection occurs. This improves the heat exchange via the conduction.

The heat exchange can be additionally improved if the catheter 23 possesses two lumens, namely an inflow 19 and an outflow 20 (see FIG. 8). In this case the medium can be exchanged within the uterus; liquid cooled or heated outside of the body can thus be continuously resupplied into the uterus while the liquid heated or cooled by heat exchange processes is removed again from the uterus via the outflow 20. In this embodiment the heat exchange occurs due to forced convection that can absorb a greater quantity of heat in comparison to the natural convection.

Independent of the number of lumens that are present, the use of a thermal insulation 29 is advantageous, as shown by FIG. 9. An interaction or a heat exchange between the liquid or the gas in the lumen and the surrounding tissue or the catheter as well can be avoided via the thermal insulation, such that the medium only begins the heat exchange in the region of the surrounding tissue. Irritation of the tissue surrounding the respective lumen (for example the cervical channel) can thereby be avoided. Moreover, the heat exchange is also concentrated at the tissue surrounding the tissue 11 to be ablated.

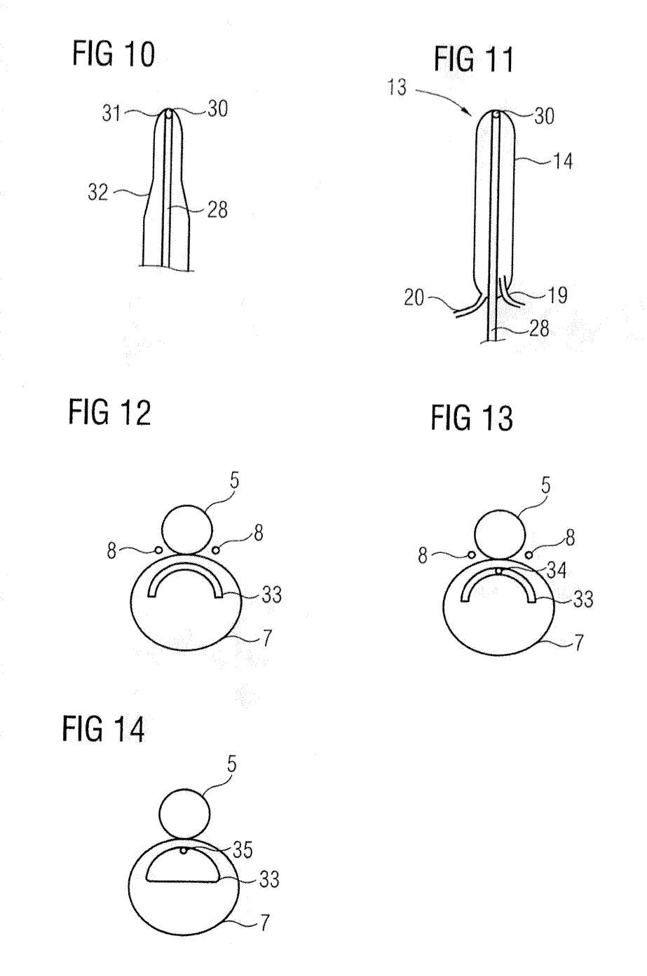

FIG. 10 shows a catheter 23 in a further embodiment, wherein a conically diverging segment 32 is provided to seal the cervical channel instead of the balloon 24 at the catheter 23. A nozzle 30 via which the medium exits from the catheter is located at the end of the lumen 28. Two cooling mechanisms can be realized by means of the nozzle 30. On the one hand, a liquid can be sprayed intermittently from the nozzle 30 so that it atomizes before striking the surrounding tissue and cools due to this adiabatic expansion. The liquid cooled in such a manner then wets the surrounding tissue and cools this.

However, the nozzle can also be designed so that the liquid exits from the catheter 23 and strikes the uterus wall without atomization. A liquid film then forms on the uterus wall that vaporizes via the heating of the surrounding tissue. Upon vaporization of the liquid film, heat is drawn from the surrounding tissue, whereby this cools.

FIG. 11 shows a balloon 13 that likewise possesses a lumen with a nozzle 30 in order to realize the cooling techniques of adiabatic expansion and the vaporization by means of the balloon 13. The balloon 13 can naturally also be inserted into the cervical channel in order to cool or warm the uterus 21. The inflow 19 and the outflow 20 are provided to fill the balloon itself, wherein a heat exchange with the surrounding tissue can also occur via the balloon 13. This naturally depends on the temperature of the medium introduced into the balloon 13.

The different warming and cooling mechanisms that are physically known can thus be realized depending on the design of the balloon 13, catheter 23 or the hollow needles 9 and 12. Whether a heat exchange occurs due to convection, conduction, vaporization or chemical reaction thus depends not on the device itself that is used but rather on the respective embodiment. The hollow needles 9 and 12 can also naturally possess a nozzle 30 at the end at the body in order to wet the inner wall of the bladder, for example, in order to achieve a cooling of the inner bladder wall via either an adiabatic expansion or via vaporization.

In addition to a medium in the form of a liquid or a gas or one of the additional aforementioned embodiments, a metal plate 33 can also be used as a heating or cooling arrangement, as FIG. 12 shows. The metal plate 33 is on the one hand adapted to the cavity into which it is inserted; on the other hand, it is optimized for heat exchange processes. In the simplest case, the metal plate 33 (which can have the most varied shapes and does not have to be fashioned only as a cuboid) is inserted into the cavity. For example, the insertion into the rectum 7 is possible given a thermal ablation of the prostate 5. Depending on the usage purpose the metal plate 33 is cooled or heated before the insertion into the rectum 7; in the rectum 7 heat exchange occurs until the surrounding tissue and the metal plate 33 have reached a common mixing temperature. After achieving the mixing temperature the metal plate 33 no longer has any heating or cooling effect; given additional heating or cooling of both the tissue 11 to be ablated and the surrounding tissue, the metal plate 33 is then also heated or cooled as well.

FIGS. 13 and 14 show how the heating or cooling effect of the metal plate 33 can also be maintained over long periods of time. For this either a Peltier cooler 34 or a heatable resistor 35 via which heat can be steadily removed from or introduced into the metal plate 33 is located in the metal plate 33. In this way a cooling or heating effect can be maintained over the entire duration of the ablation, even given ablation treatments lasting for longer periods of time.

As described above, how long the additional cooling or heating arrangement cools or heats the surrounding tissue depends on its initial temperature, its mass and its specific heat capacity. These determine how much heat the additional cooling or heating arrangement can draw from or supply to the surrounding tissue until a mixture temperature is achieved. If, due to the ablation via the first heating or cooling device, the heat introduced into or extracted from the surrounding tissue is greater than the amount of heat that can be extracted or introduced by the additional cooling or heating arrangement, an additional cooler (for example a Peltier cooler 24) or an additional heater (for example a heatable resistor 35) is to be provided. Given the presence of an additional cooler or heater, the metal plate 33 is also not necessarily to be cooled or heated before the insertion into the body.

The heating or cooling of the additional cooling or heating arrangement—regardless of whether it is thereby a metal plate 33, the balloon 13 filled with the liquid 17, the catheter 23 or the hollow needles 9 and 12—thereby naturally relates to the temperature difference between the surrounding tissue and the additional cooling or heating arrangement. Temperature differences between the surrounding tissue and the additional cooling or heating arrangement that is heated before the insertion into the body of the patient 6 that are too large must be avoided since otherwise this can lead to damage to the surrounding tissue by the additional cooling or heating arrangement. In particular, a temperature difference of less than 5° C. is suitable to cool or to heat the surrounding tissue without damaging it.

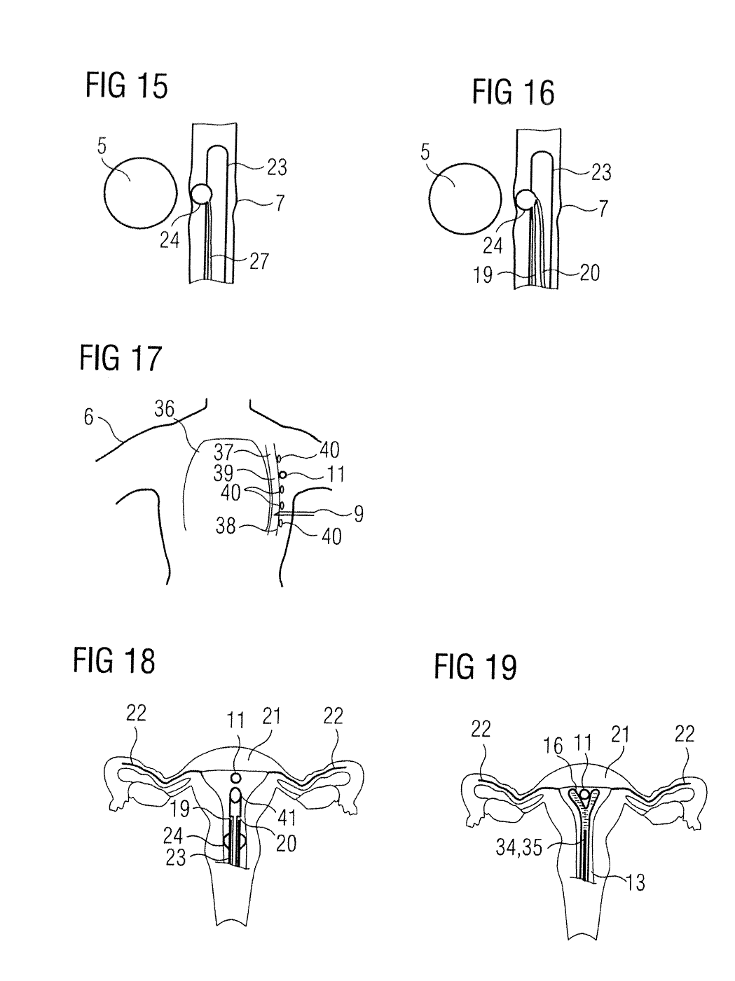

FIGS. 15 and 16 show a possible combination of a balloon 24 with a catheter 23 in which the balloon 24 is used as an additional cooling or heating arrangement. The catheter 23 is inserted into the rectum 7 in order to protect the wall of the rectum 7. After the catheter 23 has been positioned, the balloon 24 is filled via the lumen 27 with a cooled or heated medium. In this embodiment the heat exchange occurs via natural convection. In this embodiment the balloon 24 does not completely encompass the catheter 23; rather, it is attached only on one side of the catheter 23. Naturally, in combination with the catheter 23 the balloon 24 can also has varying wall thicknesses, wherein the segment 16 with the thicker wall thickness is facing towards the catheter 23.

FIG. 16 shows that a forced convection for improved cooling of the wall of the rectum 7 can also be conducted given this combination of catheter 23 and balloon 24. For this the inflow 19 and the outflow 20 are connected with the internal space of the balloon 24 in order to produce a circulation of the medium in this.

In addition to the insertion of the cooling or heating arrangement into natural cavities of the human body, it is also possible for the additional cooling or heating arrangement to be inserted into a latent cavity. Such a latent cavity is, for example, the pleural cavity 39. This surrounds the lung tissue 36 and is arranged between the visceral pleura 37 and parietal pleura 38. The ribs 40 are located outside of the parietal pleura. To protect the rib tissue, a heated liquid is inserted into the pleural cavity 39 with the hollow needle 9 in order to protect the lung tissue 36 given a cryo-ablation of the tissue 11 to be ablated. Instead of a hollow needle 9, two hollow needles 12 can also be arranged, respectively one below and one above the tissue 11 to be ablated. In this way a forced convection via which the lung tissue 36 is even better protected can be caused between the hollow needles 12.

In principle the thermal ablation device and the additional cooling or heating arrangement are objects arranged at a physical distance.

The cooling or heating arrangement can consist of a medium and a device, wherein the device introduces the medium into the cavity of the patient 6. In this case the ablation device 1 and the device to introduce the medium are advantageously combined into a single device. FIG. 18 shows an exemplary embodiment in which a catheter 23 is inserted into the uterus 21. To ablate the tissue 11 to be ablated, a cryo-unit 41 with which the tissue 11 to be ablated is obliterated by removing heat is located at the tip of the catheter 23. To protect the uterus wall, the catheter has two lumens that act as an inflow 19 and outflow 20. A forced convection of the introduced medium is achieved in the uterus via the lumens, wherein the uterus wall is heated by the medium introduced via the inflow 19. The cryo-unit thus primarily develops its effect at the tissue 11 to be ablated while a damage to the surrounding tissue—namely the uterus wall—can be avoided.

FIG. 19 shows a possibility to also achieve a temporally persistent cooling or heating of a fluid in a balloon 13 without forced convection. For this a Peltier cooler 34 or a heatable resistor 35 or even both a Peltier cooler 34 and a heatable resistor 35 simultaneously is/are located in the balloon 13. The liquid 17 inside the balloon 13 is cooled or heated by setting either the Peltier cooler or the heatable resistor 35 into operation. Heat can thereby be removed or, respectively, supplied over an arbitrary time period to the tissue surrounding the tissue 11 to be ablated without using an inflow and an outflow. The balloon 13 can be used for all thermal ablation techniques via the presence of a respective cooler and heater. It furthermore arises from FIG. 19 that the balloon 13 can also adopt other shapes than the shape already described. In particular, a structure that is bifurcated as viewed in cross section is also possible, wherein in this case the inner wall is fashioned as a segment 16 with a thicker wall thickness. The heat exchange is thereby minimized between the tissue 11 to be ablated and the balloon 13.

Although modifications and changes may be suggested by those skilled in the art, it is the intention of the inventors to embody within the patent warranted hereon all changes and modifications as reasonably and properly come within the scope of their contribution to the art.

We claim as our invention:

1. A thermal ablation device comprising:
a first temperature modifying device configured to interact in vivo with tissue in a treatment region to modify the temperature of said tissue in order to ablate said tissue; and
at least one second temperature modifying device configured to interact with tissue surrounding said tissue in said treatment region to modify the temperature of said tissue surrounding said treatment region, said second temperature modifying device being configured for introduction into a cavity in the patient selected from the group consisting of natural cavities and latent cavities.

2. An ablation device as claimed in claim 1 wherein said second temperature modifying device has a larger heat capacity than said tissue surrounding said treatment region.

3. An ablation device as claimed in claim 1 wherein said second temperature modifying device is a cooling device and, upon introduction of said cooling device into said body, said cooling device having a temperature that is lower than the body temperature of the patient.

4. An ablation device as claimed in claim 3 wherein said cooling device has a temperature that is less than 5° C. from said body temperature of the patient.

5. An ablation device as claimed in claim 1 wherein said second temperature modifying device is a heating device and, upon introduction of said heating device into said body, said heating device having a temperature that is higher than the body temperature of the patient.

6. An ablation device as claimed in claim 5 wherein said cooling device has a temperature that is than 5° C. from said body temperature of the patient.

7. An ablation device as claimed in claim 1 wherein said second temperature modifying device is configured to supply a fluid medium to said tissue surrounding said treatment region to modify the temperature of said tissue surrounding said treatment region.

8. An ablation device as claimed in claim wherein said second temperature modifying device comprises a balloon filliable with said medium.

9. An ablation device as claimed in claim 8 wherein said balloon has a balloon wall having a non-uniform wall thickness,

10. An ablation device as claimed in claim 9 wherein said balloon comprises a circular thinning of said wall thickness.

11. An ablation device as claimed in claim 1 wherein said second temperature modifying device comprises at least one hollow needle configured for insertion in said tissue surrounding said treatment region,

12. An ablation device as claimed in claim 1 wherein said second temperature modifying device is configured as a catheter.

13. An ablation device as claimed in claim 12 wherein said catheter comprises a balloon located at a distal region of said catheter, said balloon being configured to seal a natural cavity in the body of the patient,

14. An ablation device as claimed in claim 12 wherein said catheter comprises a plurality of openings at distal end thereof for introduction of a fluid medium into said cavity in the body of the patient.

15. An ablation device as claimed in claim 5 wherein said second temperature modifying device comprises a balloon filled with a fluid medium that interacts with said tissue surrounding said treatment region to modify the temperature of said tissue surrounding said treatment region, and wherein said second temperature modifying device further comprises at least one inflow and at least one outflow in fluid communication with an interior of said balloon allowing exchanging of said fluid medium in said balloon.

16. An ablation device as claimed in claim 15 wherein said second temperature modifying device is configured as a catheter, and wherein said catheter comprises a first lumen forming said inflow and a second lumen forming said outflow.

17. An ablation device as claimed in claim 16 comprising thermal insulation that insulates said first and second lumens.

18. An ablation device as claimed in claim 1 wherein said second temperature modifying device comprises a balloon having an interior wall, and a nozzle that sprays a fluid medium onto said interior wall of said balloon, said balloon interacting with said tissue surrounding said treatment region and said fluid medium sprayed on said wall in said balloon modifying the temperature of said tissue surrounding said treatment region.

19. An ablation device as claimed in claim 1 wherein said second temperature modifying device comprises a nozzle opening into said cavity, said nozzle spraying a fluid medium into said cavity that modifies the temperature of the tissue surrounding said treatment region.

20. An ablation device as claimed in claim 1 wherein said second temperature modifying device comprises a chemical reagent that releases or extracts heat by a chemical reaction, said second temperature modifying device being configured to release said heat into or extract said heat from said tissue surrounding said treatment region.

21. An ablation device as claimed in claim 1 wherein said second temperature modifying device comprises a metal plate.

22. An ablation device as claimed in claim 1 wherein said second temperature modifying device comprises a Peltier cooler, and a fluid medium cooled by said Peltier cooler, and wherein said second temperature modifying device is configured to cause said fluid medium cooled by said Peltier cooler to interact with said tissue surrounding said treatment region.

23. An ablation device as claimed in claim 1 wherein said second temperature modifying device comprises a heatable resistor, and a fluid medium heated by said heatable resistor, and wherein said second temperature modifying device is configured to cause said fluid medium heated by said heatable resistor to interact with said tissue surrounding said treatment region.

24. An ablation device as claimed in claim 1 wherein said second temperature modifying device comprises a liquid medium and wherein said second temperature modifying device is configured to deliver said liquid medium as a liquid film on a wall of said cavity.

25. An ablation device as claimed in claim 1 wherein said second temperature modifying device comprises a balloon that expands into said cavity, said balloon having an interior wall, and said second temperature modifying device comprising a liquid medium formed as a liquid film on said interior wall of said balloon, said liquid film on said interior of said balloon interacting with said tissue surrounding said treatment region to modify the temperature of said tissue surrounding said treatment region.

26. A thermal ablation catheter comprising:
a catheter configured for in vivo introduction into the body of a patient;
said catheter comprising a first temperature modifying device configured to interact in vivo with tissue in a treatment region to modify the temperature of said tissue in order to ablate said tissue; and
said catheter comprising at least one second temperature modifying device configured to interact with tissue surrounding said tissue in said treatment region to modify the temperature of said tissue surrounding said treatment region, said second temperature modifying device being configured for introduction into a cavity in the patient selected from the group consisting of natural cavities and latent cavities.

27. A thermal ablation catheter as claimed in claim 26 wherein said first temperature modifying device is located at a distal end of said catheter.

28. A thermal ablation catheter as claimed in claim 26 comprising a balloon that is variable in volume, and a lumen proceeding through said catheter and being in fluid communication with an interior of said balloon to fill said interior of said balloon with a fluid medium that interacts with said tissue surrounding said treatment region to modify the temperature of said tissue surrounding said treatment region.

29. A thermal ablation catheter as claimed in claim 28 comprising a first lumen in said catheter and a second lumen in said catheter, said first lumen providing an inflow of said fluid medium to said interior of said balloon and said second lumen providing an outflow of said fluid medium from said interior of said balloon, allowing said fluid medium in said interior of said balloon to be exchanged.

30. A thermal ablation catheter as claimed in claim 29 comprising thermal insulation that thermally insulates said first lumen and said second lumen.

31. A thermal ablation catheter as claimed in claim 26 wherein said second temperature modifying device comprises a balloon that is variable in volume and a first lumen in said catheter in fluid communication with an interior of said balloon, said first lumen filling said interior of said balloon with a first fluid, and said catheter comprising a second lumen that discharges into said cavity in the body of the patient, said second lumen delivering a second fluid to said natural cavity.

32. A method for thermally ablating tissue, comprising the steps of:
introducing a first temperature modifying device to interact in vivo with tissue in a treatment region to modify the temperature of said tissue in order to ablate said tissue; and
introducing at least one second temperature modifying device to interact with tissue surrounding said tissue in said treatment region to modify the temperature of said tissue surrounding said treatment region, by introducing said second temperature modifying device into a cavity in the patient selected from the group consisting of natural cavities and latent cavities.
